(19) United States
(12) Patent Application Publication
(10) Pub. No.: US 2024/0061226 A1
(43) Pub. Date: Feb. 22, 2024

(54) MULTIPHASE OPTICAL COHERENCE MICROSCOPY IMAGING

(71) Applicant: The Board of Trustees of the University of Illinois, Urbana, IL (US)

(72) Inventors: Stephen A. Boppart, Champaign, IL (US); Mantas Zurauskas, Urbana, IL (US)

(21) Appl. No.: 18/260,987

(22) PCT Filed: Jan. 12, 2022
(86) PCT No.: PCT/US22/12211

§ 371 Date: Jul. 11, 2023

Related U.S. Application Data
(60) Provisional application No. 63/136,624, filed on Jan. 12, 2021.

(51) Int. Cl. G02B 21/00; G01B 9/02091

(52) U.S. Cl. CPC G02B 21/0056; G01B 9/02091; G01B 2290/70

(57) ABSTRACT

Systems and methods for phase-sensitive optical coherence tomography (“OCT”) and/or optical coherence microscopy (“OCM”) include imparting multiple phase shifts to the object beam, the reference beam, or both in an interferometer. Phase shifts can be imparted to the S-polarization and/or P-polarization modes of the object and/or reference beams. As an example, phase shifts can be imparted to the reference beam in the reference arm using a waveplate or other phase shifter. A non-polarizing beamsplitter can provide additional phase shifts to light reflected by the beamsplitter. Full field OCM can be provided by imaging the phase-shifted channels using an image sensor.

### CROSS-REFERENCE TO RELATED APPLICATIONS

This application claims the benefit of U.S. Provisional Patent Application Ser. No. 63/136,624, filed on Jan. 12, 2021, and entitled “Multiphase Optical Coherence Microscopy Imaging,” which is herein incorporated by reference in its entirety.

### STATEMENT OF FEDERALLY SPONSORED RESEARCH

This invention was made with government support under FA9550-17-1-0387 awarded by the U.S. Air Force Office of Scientific Research. The government has certain rights in the invention.

### BACKGROUND

Label-free imaging is re-emerging as the ultimate approach for observing cells and living organisms in their natural state. Optical coherence microscopy (“OCM”) is a label-free imaging technique based on low coherence interferometry. It is particularly well suited for label-free biomedical imaging as it can provide phase, amplitude and polarization information while using incoherent illumination with low average and low peak power illumination.

Full field OCM (“FF-OCM”) imaging employs a camera for detection of intensity images of interferograms, formed by interference of the light backscattered from an object and reference arms of an imaging system. Coherence gating permits decoupling of axial and lateral resolution, and allows the rejection of light originating from outside of the coherence gate, which is defined by the optical spectrum of illumination source is used for imaging. Time domain FF-OCM (“TD-FF-OCM”) is one of the fastest implementations of FF-OCM, permitting observation of single en-face imaging plane at rates limited by a camera framerate. Typically to reconstruct TD-FF-OCM image containing phase and intensity information at least three interferograms with different phase shifts between the object and reference arms are required. It has been long known that for rapid and robust implementation of TD-FF-OCM, interferograms required for image reconstruction should be captured simultaneously.

Phase sensitive coherence gated imaging can provide multiple additional benefits, beyond optical sectioning. One advantage of phase sensitive OCM is that it permits observing sample displacements on a scale several order of magnitude lower than axial resolution, down to sub-nanometer scale. If sufficient phase stability can be achieved, phase sensitive coherence gated imaging unlocks new applications in angiography, neuro-imaging, and other fields. Additionally, complex images captured with stable phase sensitive imaging systems can benefit from correction with computational adaptive optics.

Single-shot phase-sensitive OCM has been implemented either by using holographic coherence (off-axis) imaging, or spatially separated phase stepped imaging. While being simple to implement, off-axis configurations often rely on spatially coherent illumination and often require compromising between resolution and dynamic range. Alternatively, FF-OCM implementations relying on geometrical phase shifts can provide achromatic phase shifts across a broad spectrum and can work with spatially incoherent illumination. However, the configuration presented to date allow for only a single orientation of linearly polarized light returning from the object arm to be detected, with other polarization states discarded by the use of a polarizing beamsplitter between the object and reference arms.

### SUMMARY OF THE DISCLOSURE

The present disclosure addresses the aforementioned drawbacks by providing an optical coherence microscopy system that includes a light source, a beamsplitter, a reference arm, and an object arm. The light source is configured to produce a light beam, where the light beam comprises S-polarized light and P-polarized light. The beamsplitter is configured to split the light beam into a reference beam and an object beam, where the reference beam comprises an S-polarized reference beam component (RS) and a P-polarized reference beam component (RP) and the object beam comprises an S-polarized object beam component (OS) and a P-polarized object beam component (OP). The reference arm is configured to receive the reference beam from the beamsplitter, impart a phase shift to at least one of the S-polarized reference beam component or the P-polarized reference beam component, and return the reference beam to the beamsplitter. The object arm is configured to receive the object beam from the beamsplitter and return the object beam to the beamsplitter (e.g., after reflecting or otherwise interacting with an object). The beamsplitter is further configured to combine the reference beam returned from the reference arm with the object beam returned from the object arm to output a phase-shifted light beam comprising a plurality of phase-shifted light beam components.

It is another aspect of the present disclosure to provide an optical assembly for use in an interferometer. The optical assembly includes a beamsplitter configured to extract a reference beam from a light beam when the light beam firstly propagates through the beamsplitter, and a reference arm. The reference arm is configured to receive the reference beam and impart a phase shift thereto before returning the reference beam to the beamsplitter, wherein the reference arm comprises a phase shifter and a reference reflector. The beamsplitter integrates the reference beam returned from the reference arm into the light beam when the reference beam propagates through the beamsplitter.

It is still another aspect of the present disclosure to provide a method for multiphase optical coherence microscopy. A reference beam and an object beam are extracted from a light beam, where the object beam includes a P-polarized object beam component (OP) and an S-polarized object beam component (OS), and the reference beam includes a P-polarized reference beam component (RP) and an S-polarized reference beam component (RS). A phase of at least one of the RS component or the RP component of the reference beam is shifted, and the reference beam and the object beam are recombined to obtain distinct phase shifts between the object beam and the reference beam in at least one of the S-polarization or P-polarization component.

The foregoing and other aspects and advantages of the present disclosure will appear from the following description. In the description, reference is made to the accompanying drawings that form a part hereof, and in which there is shown by way of illustration one or more embodiments. These embodiments do not necessarily represent the full scope of the invention, however, and reference is therefore made to the claims and herein for interpreting the scope of the invention.

### BRIEF DESCRIPTION OF THE DRAWINGS

FIG. 1 shows an example phase shifting strategy in accordance with some embodiments of the present disclosure. In FIG. 1, unpolarized light from light source can be described as sum of two orthogonally polarized components S and P. The light is divided into object (OS and OP) and reference (RS and RP) arms. In the reference arm, the component RP is delayed to impart a phase shift. After reflection from the beamsplitter, the reflected components RS and RP undergo a further phase shift in relation with transmitted light. This results in four distinct phase shifts between the light returned from object and the reference arm.

FIG. 2 shows an example multiphase optical coherence microscopy system in accordance with some embodiments described in the present disclosure.

FIG. 3 is a flowchart setting forth the steps of an example method for multiphase optical coherence microscopy.

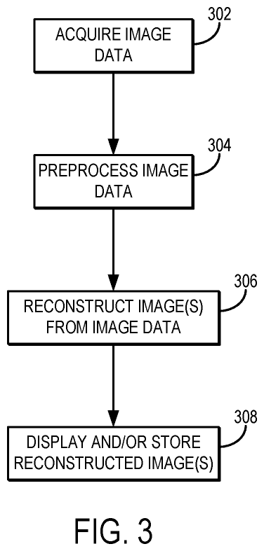

FIGS. 4A-4C show an example of 4-phase OCM imaging in accordance with an embodiment of the present disclosure. a) Four simultaneously captured interferograms from a flat mirror used as a sample. b) The intensity profiles from the interferograms depicted in a), showing that the phase in each channel is shifted by the expected step of π/2 radians. c) Urea crystals grown on a glass coverslip. Images show one of the raw channels used for reconstruction (left), as well as the intensity (center) and phase (right) of the OCM reconstructions for two axial locations.

FIGS. 5A-5E show phase sensitive imaging in accordance with an embodiment of the present disclosure. Complex images of a calibration target demonstrate high dynamic range and stability of the set-up. Images a), b), and c) depict amplitude and phase components of the image. The scale bars represent 50 d) and e) Phase stability measurements over time before and after, respectively, computational correction of global drift.

FIGS. 6A-6D show phase sensitive OCM imaging of live fibroblast cell cultures in accordance with an embodiment of the present disclosure. Imaging the surface of a collagen substrate to which the cells are attached permits sensing cumulative optical path differences induced by the cells. FIG. 6A shows the intensity component and FIG. 6B shows the phase component. The phase image is unwrapped (FIG. 6C) and flattened to produce quantitative images indicating the optical path delay induced by the cell (FIG. 6D). Representative images are shown with no averaging, captured at a 500 Hz framerate. Scale bar represents 20 μm.

FIGS. 7A-7E show dynamic imaging of live macrophage cells reveal metabolic activity at the sub cellular level. FIG. 7A show the average of intensity of OCM reconstructions. FIG. 7B shows the average temporal frequency content (average of pixel-wise FFT with DC component excluded). FIG. 7C shows the variance of the frequency content. Frequency analysis produces higher contrast between the cell nuclei and cytoplasm if longer image sequences are analyzed—500 s in FIG. 7E. However, for shorter sequences captured at a 500 Hz rate over 1 s as shown in FIG. 7D, individual organelles become more pronounced (white arrows).

FIG. 8 is a block diagram of an example computer system that can implement the methods described in the present disclosure.

### DETAILED DESCRIPTION

Described here are systems and methods for phase-sensitive optical coherence tomography (“OCT”). In some embodiments, the systems and methods described in the present disclosure enable phase-sensitive optical coherence microscopy (“OCM”). In general, the systems and methods described in the present disclosure enable multiphase OCT, which may include phase-sensitive single-shot time-domain OCM, multiphase full-field (“FF”) OCM, and the like. Advantageously, due to their inherent resistance to speckle noise and optical aberrations the systems and methods can enable FF-OCM with high spatial and temporal resolution.

In some embodiments, the systems and methods described in the present disclosure enable FF-OCT and/or FF-OCM, which includes capturing single-shot phase-sensitive imaging through simultaneous acquisition of three or more phase-shifted images with a single camera using unpolarized light for object illumination. Advantageously, in some embodiments the full dynamic range of the camera can be retained by using different areas of a single camera sensor to capture each image.

As will be described in more detail, in some embodiments, a system for multiphase OCM imaging includes a light source, a beamsplitter, a waveplate, and a reference mirror. The light source emits a light beam that is incident on the beamsplitter. The beamsplitter extracts a reference beam, which includes a P-polarized reference beam element, from the light beam when the light beam propagates through the beamsplitter. The reference beam propagates through a waveplate and is then incident on a reference mirror. The reference mirror reflects the reference beam such that the reference mirror propagates again through the beamsplitter, and the beamsplitter integrates the reference beam into the light beam.

The beamsplitter may also extract an object beam from the light beam when the light beam propagates through the beamsplitter. The object beam is incident on an object mirror, microscopy interface, or other object that reflects the object beam such that it propagates again through the beamsplitter, and the beamsplitter integrates the object beam into the light beam.

The reference beam may further include an S-polarized reference beam element, and the beamsplitter shifts the phase of the S-polarized reference beam element when the reference beam propagates again through the beamsplitter. The waveplate may shift the phase of the P-polarized reference beam element by π/2, and the beamsplitter may shift the phase of the P-polarized reference beam element by π.

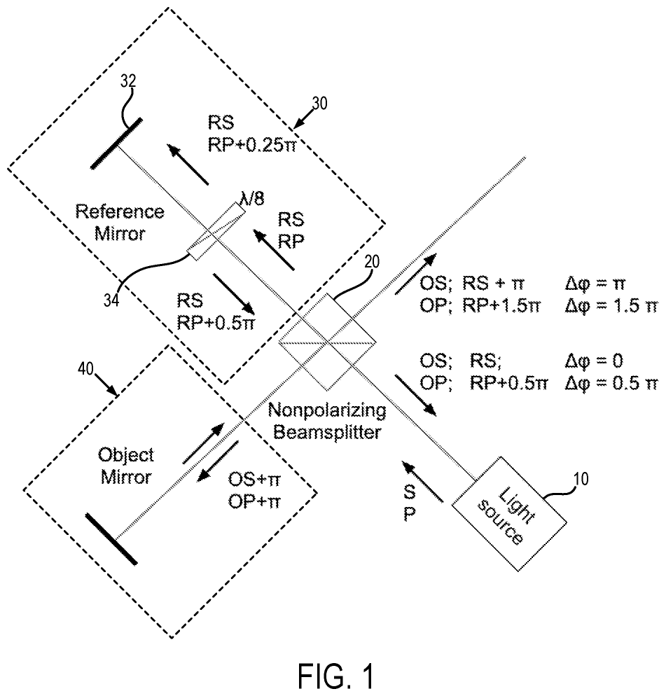

Referring now to FIG. 1, an example phase shifting scheme employed by some embodiments of the systems and methods described in the present disclosure is illustrated. Light is emitted by a light source 10. The light source 10 is preferable an unpolarized light source, such that the emitted light beam has similar intensity in both the S-polarized and P-polarized modes. As shown, the light beam thus contains an S-polarized light beam component (“S”) and a P-polarized light beam component (“P”). The light beam is propagated through a beamsplitter 20 and split into a reference beam that propagates into a reference arm 30 and an object beam that propagates into an object arm 40. The reference beam contains an S-polarized reference beam component (“RS”) and a P-polarized reference beam component (“RP”), and the object beam contains an S-polarized object beam component (“OS”) and a P-polarized object beam component (“OP”).

The reference arm 30 contains a reference reflector 32 (e.g., a reference mirror) and a phase shifter 34. The phase shifter 34 can be a waveplate, such as a λ/8 waveplate, λ/4 waveplate, or the like. In some embodiments, the waveplate may be an achromatic waveplate. As the reference beam passes through the phase shifter 34, a phase shift is imparted to the reference beam. For example, a phase shift can be imparted to the RS component, the RP component, or both. In the illustrated embodiment, a λ/8 waveplate imparts a π/4 phase shift to the RP component on each pass through the waveplate, resulting in a π/2 phase shift to the RP component as it exits the reference arm.

The beamsplitter 20 is preferably a non-polarizing beamsplitter. The beamsplitter 20 is configured to impart an additional phase shift to the reference beam returned from the reference arm 30. The phase shifts at the beamsplitter 20 can be varied based on the beamsplitter 20 design. As an example, the beamsplitter 20 can be a 50:50 cube beamsplitter composed of two triangular prisms with a dielectric coating applied to the hypotenuse of one of the two triangular prisms. In the illustrated embodiment, the light phase is shifted only during the reflection after the reference beam is returned from the reference arm 30 and/or as the object beam is split into the object arm 40.

The additional phase shift from the beamsplitter 20 can be imparted to both the reference beam and the object beam. For instance, the additional phase shift can be imparted to the RS component of the reference beam, the RP component of the reference beam, the OS component of the object beam, the OP component of the object beam, or combinations thereof. In the illustrated embodiment, an additional phase shift of π is imparted to both the OS component and the OP component reflected by the beamsplitter 20 from the light source 10 into the object arm 40, and an additional phase shift of π is imparted to both the RS component and the RP component reflected by the beamsplitter 20 after the reference beam is returned from the reference arm 30. In the configuration of the illustrated embodiment, four phase shifts, stepped by of π/2, are obtained between the object and reference beams in the S and P polarization modes, as shown in FIG. 1. These phase-shifted channels can then be spatially separated using a polarizing beamsplitter and recorded using a single camera as described below.

The phase shifting approach allows achromatic performance, which is advantageous when using broadband light sources in OCM, as the beamsplitter-induced phase shift is geometric. Additionally, the phase shift(s) induced by the phase shifter 34 can also be achromatized through the use of achromatic waveplates. The phase shifts achieved using the systems and methods described in the present disclosure are stable due to the absence of moving parts, permitting accurate OCM reconstructions.

In some embodiments, beamsplitter 20 and optics in the reference arm 30 (e.g., the phase shifter 34) form an optical assembly that is configured to impart multiple different phase shifts (e.g., three or more phase shifts) to light for use in optical coherence tomography applications, which in some instances may include optical coherence microscopy applications as described. It will be appreciated by those skilled in the art that this optical assembly (e.g., the beamsplitter 20 and phase shifter 34 designed to impart multiple different phase shifts to light passing therethrough) can advantageously be used in optical systems other than those expressly described in the present disclosure.

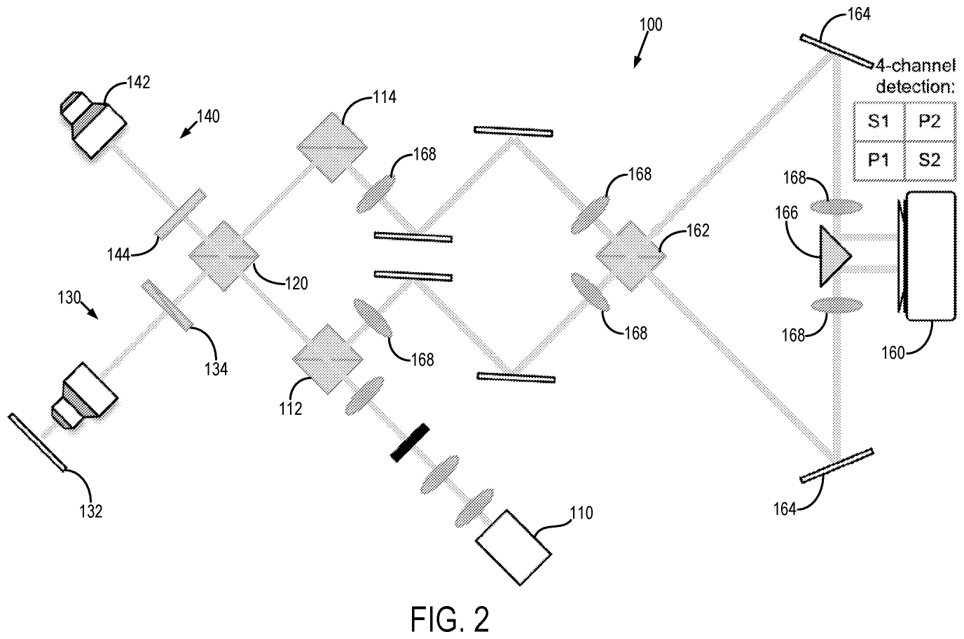

FIG. 2 illustrates an example optical coherence microscopy system 100 that implements the systems and methods described in the present disclosure. The optical coherence microscopy system 100 includes an interferometer portion, which is generally composed of a light source 110, a beamsplitter 120, a reference arm 130, and an object arm 140. The light source 110 produces a light beam that is split by the beamsplitter 120 into a reference beam that illuminates optics in the reference arm 130, and an object beam that illuminates optics in the object arm 140. The reference beam returned from the reference arm 130 and the object beam returned from the object arm 140 are recombined in a second portion of the optical coherence microscopy system 100 and are spatially separated and recorded using an image sensor 160, which in some examples may be a camera.

As described above, the light source 110 is preferably an unpolarized light source. As a non-limiting example, the light source 110 can be a light emitting diode (“LED”), a halogen bulb, or the like. Additionally or alternatively, the light source may include other light source, such as a super-luminescent diode (“SLD”), a supercontinuum light source, another broadband light source, a light source having random phase, or the like. The light source 110 is preferably also a low-coherence or otherwise spatially incoherent light source. Advantageously, LEDs, halogen bulbs, and other light sources can provide unpolarized, low-coherence light, which may in some implementations also be broadband. As a non-limiting example, the light source 110 can be an LED with a central wavelength of 565 nm and a spectral full width at half max (“FWHM”) of 104 nm. Sufficient uniformity of the light source 110 emission enables critical illumination (i.e., Nelsonian illumination), where the source is imaged on the sample, thereby preserving the spatial incoherence of the source. For example, critical illumination can be implemented by magnifying and imaging the active area of the light source 110 onto the field aperture of the system.

Light can be injected into the optical coherence microscopy system 100 through a first non-polarizing beamsplitter 112. For example, the non-polarizing beamsplitter 112 can be a 70:30 (R:T) non-polarizing beamsplitter. The illumination light beam is then split into the reference arm 130 and the object arm 140 by a second non-polarizing beamsplitter 120.

As described above, the second non-polarizing beamsplitter 120 splits the illumination light beam into a reference beam propagating in the reference arm 130, and an object beam propagating in the object arm 140. The reference beam is composed of RS and RP components, and the object beam is composed of OS and OP components, as described above. As a non-limiting example, the second non-polarizing beamsplitter 120 can be a 50:50 non-polarizing beamsplitter cube. For instance, the second non-polarizing beamsplitter 120 can be a 50:50 cube beamsplitter composed of two triangular prisms with a dielectric coating applied to the hypotenuse of one of the two triangular prisms. The construction of the second non-polarizing beamsplitter 120 can be configured to impart a specific phase shift to light reflected by the second non-polarizing beamsplitter 120. For example, the second non-polarizing beamsplitter 120 can be constructed or otherwise designed to impart a π phase shift, a π/2 phase shift, a π/4 phase shift, and so on.

The second non-polarizing beamsplitter 120 is configured such that an additional phase shift is imparted to the reference beam and/or object beam upon reflection at the second non-polarizing beamsplitter 120. For instance, when the object beam is first reflected by the second non-polarizing beamsplitter 120 into the object arm, a phase shift can be imparted to the OS component, the OP component, or both. Upon exiting the object arm 140, the object beam is split into a first and second object beam. The first object beam part is transmitted through the second non-polarizing beamsplitter 120 and thus does not accrue and additional phase shift. The second object beam part is reflected by the second non-polarizing beamsplitter 120 and thus accrues an additional phase shift.

As a non-limiting example, the second non-polarizing beamsplitter 120 can be configured to impart a π phase shift, such that in the described example the first object beam part would have a it phase shift—having been phase shifted only once—and the second object beam part would have a 2π phase shift (i.e., returned to the original light phase). Similarly, when the reference beam returned from the reference arm 130, the second non-polarizing beamsplitter 120 will reflect a portion of the reference beam as a first reference beam part and allow transmission of a second portion of the reference beam as a second reference beam part. The first reference beam part (i.e., the reflected portion) will accrue a phase shift from the second non-polarizing beamsplitter 120. For instance, in the non-limiting example above, the first reference beam part would accrue an additional π phase shift when reflected by the second non-polarizing beamsplitter 120 (in addition to the phase shifts imparted by the waveplate 134 in the reference arm 130) and the second beam part would not accrue an additional phase shift, but instead would include the phase shifts imparted by the waveplate 134 in the reference arm 130.

The reference arm 130 includes a reference reflector 132 (e.g., a reference mirror) and a phase shifter, which in the illustrated embodiment includes a waveplate 134, such as a λ/8 waveplate, or the like. In some implementations, the waveplate 134 may be a continuously variable waveplate, such as a Soleil-Babinet compensator (“SBC”). When using an SBC, it may be possible that the real phase shifts achieved will vary slightly for different wavelengths. Thus, in some other embodiments, the waveplate 134 can implement a fully achromatic phase shifting strategy, such as where the internal reflection from a prism hypotenuse surface is used to achieve wavelength-independent shifts. The reference beam first passes through the waveplate 134 before being reflected by the reference reflector 132 back through the waveplate 134 a second time and directed towards the beamsplitter 120.

The object arm 140 includes a microscopy interface 142 for directing the object beam onto an object and for receiving light backreflected from the object as a reflected object beam. The microscopy interface 142 may include one or more objective lenses, or the like. As a non-limiting example, the microscopy interface 142 can include objective lenses mounted in an upright configuration and containing pairs of either 0.25 numerical aperture (“NA”) dry or 0.8 NA water immersion objectives.

Additionally or alternatively, the object arm 140 may include other optics, such as a dispersion compensator 144. For instance, the dispersion compensator 144 can be assembled to match dispersion introduced by the waveplate 134 used in the reference arm 130.

After backreflecting from the reference reflector 132 and from the object, the light from the reference and object beams may be recombined at the second non-polarizing beamsplitter cube 120 and then spatially separated and directed toward an image sensor 160 to record one or more images. The image sensor 160 may include a charge-coupled device (“CCD”) light sensor, a complementary metal-oxide-semiconductor (“CMOS”) light sensor, or other type of active pixel sensor, image sensor, or camera.

In the illustrated embodiment, the first non-polarizing beamsplitter 112 and a third non-polarizing beamsplitter 114 can be used to direct the returned object and reference beams towards the image sensor 160. For instance, the first non-polarized beamsplitter 112 can be used to reflect the first object beam part (i.e., the portion of the object beam reflected by the second non-polarizing beamsplitter 120) and the second reference beam part (i.e., the portion of the reference beam transmitted through the second non-polarizing beamsplitter 120 and the third non-polarizing beamsplitter 114 can be used to reflect the second object beam part (i.e., the portion of the object beam transmitted through the second non-polarizing beamsplitter 120) and the first reference beam part (i.e., the portion of the reference beam reflected by the second non-polarizing beamsplitter 120).

Steering mirrors 162 may be used to ensure that the images formed on the image sensor 160 are laterally displaced and do not overlap along the vertical axis (i.e., direction perpendicular to the drawing plane in FIG. 2). In some embodiments, a different portion of the image sensor 160 is used to record image data from each phase-shifted channel. In some other embodiments, phase-shifted channels may be partially or fully overlapped on common portions of the image sensor 160. Advantageously, using different areas of a single camera sensor 160 to capture each image allows for the full dynamic range of the camera sensor 160 to be retained.

The S and P polarization modes may be separated by a polarizing beamsplitter cube 164. Throughout the setup of the optical coherence microscopy system 100, telescopes may be assembled from lenses 168. In a non-limiting example, the lenses may be achromatic doublets with focal lengths f=150 mm. Further, the lenses 168 may be arranged in 4-f configurations, or other suitable optical relays or the like. The same lenses may also be used to form the images of the phase-shifted channels on the image sensor 160.

A prism 166, which in some embodiments may be knife-edge right-angle silver coated prism, may be used to ensure that the images are positioned close enough, but separated on the image sensor 160. In a non-limiting example, for image detection, a CMOS camera sensor may be coupled with a frame grabber (e.g., Euresys Coaxlink Quad G3).

As described above, the OCM systems described in the present disclosure can enable FF-OCM, which utilizes parallel detection of light waves that are reflected or scattered back from the sample. The object light interferes with the reference light if the optical path length is matched to within the coherence length of the light source, producing a signal that is later used to reconstruct the image of the sample.

In FF-OCM, optical aberrations or multiple scattering events can lead to incorrect mapping of the light and unintended interference signal. Therefore, spatially incoherent light sources are particularly suitable for the multiphase OCM techniques described in the present disclosure. If the sample is illuminated with a light source with low spatial and temporal coherence, the scattered or aberrated light returned from the sample becomes spatially and temporally misaligned with the reference and does not interfere. As a result, the resolution is not compromised and only signal-to-noise ratio (“SNR”) is affected. Paired with broadband, spatially incoherent light sources, multiphase OCM is robust against speckle noise. As described above, the systems and methods described in the present disclosure are compatible with all major types of light sources commonly used in FF-OCM, including SLD light sources, supercontinuum light sources, or other broadband light sources. The systems and methods described in the present disclosure are particularly well-suited for used with spatially incoherent sources, such as LEDs or other light sources with random phase.

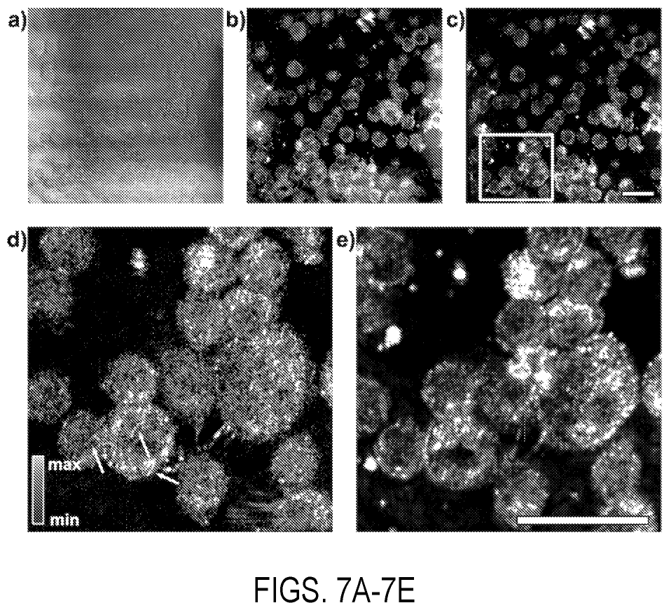

Referring now to FIG. 7, a flowchart is illustrated as setting forth an example method for imaging an object using a multiphase optical coherence microscopy system, such as those described in the present disclosure.

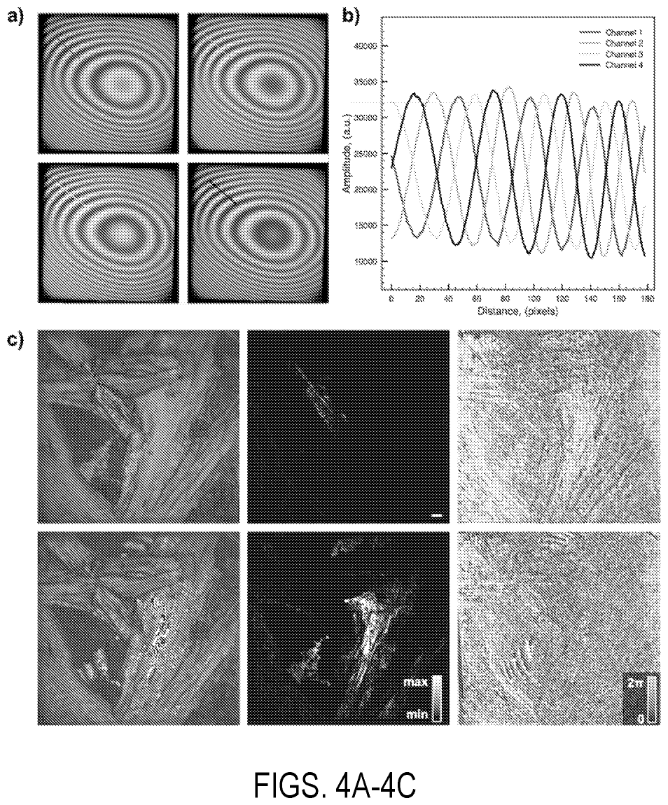

The method includes acquiring image data with the optical coherence microscopy system, as indicated at step 702. The acquired image data include multiphase image data, such as image data acquired on two or more different phase-shifted channels, such as three or four different phase-shifted channels. In a non-limiting example, four simultaneously captured phase-shifted interferograms are shown in FIG. 4. In this example, the surface of a flat silver mirror positioned at the focus of the imaging objective was used as an object. The interference bands are visible when the coherence gate is not matched with the focal plane and can be eliminated through further alignment or computational correction.

The detection sensitivity for the OCM systems described in the present disclosure at shot-noise-limited conditions can be defined as 10 log (Rₘᵢₙ), where Rₘᵢₙ is the minimum detectable reflectivity (at SNR=1). Rₘᵢₙ can be found using the following:

$${R}_{min} = 0.46 ( \frac{{ξ}_{sat} + {χ}^{2}}{N {ξ}_{sat}^{2}} ) ( \frac{{( {R}_{obj} + {R}_{ref} )}^{2}}{{R}_{ref}} ) \qquad ( 1 )$$

Here ξₛₐₜ is determined by the full well capacity of the camera sensor, which as an example with no binning can be specified as 2000000 e−; χ is the total electrical noise, which is equal to the read noise of 945 e− in the illustrated example; M=1 is gain; N=1 is the number of accumulations; and R_{obj} and R_{ref} represent the relative intensities of the light returned from the object and the reference arms, respectively. Using Eqn. (1), the sensitivity is −59 dB when imaging in conditions where object and reference reflectivity is matched.

The round trip coherence gate length can be determined by the central wavelength and bandwidth, which for the example system described above where the central wavelength is λ_{c}=565 nm and the bandwidth is Δλ=104 nm, the round trip coherence gate length of the light source can be measured to be 1+0.1 μm. The phase sensitivity, which can be determined from the standard deviation of the signal from a single pixel in 50 frames, measured using a mirror as a sample, was 1.2 mrad, corresponding to displacement sensitivity of 0.1 nm.

Advantageously, in some embodiments the full dynamic range of the camera can be retained by using different areas of a single camera sensor to capture each image, as described above.

One or more data preprocessing steps can be implemented before image reconstructions, as generally indicated at process block 704. The preprocessing steps may include correcting for geometrical distortions, reducing noise, and the like.

In an embodiment, the phase shifted images I₁₋₄ propagate through different optical paths, therefore the images formed on the camera are affected by different geometrical distortions. To correct for this, displacement maps can be obtained. In an example embodiment, after each system re-alignment, an image of a reflective structure with sufficient spatial structure (e.g., a flat surface of sand-blasted steel) can be captured with the reference arm blocked. This frame cam ne used to extract four imaging channels I₁₋₄. Image I₁ from the first imaging channel can be used as a reference to calculate the displacement maps for other three channels. As a non-limiting example, the displacement maps can be calculated using an image registration algorithm, such as a non-parametric diffeomorphic image registration algorithm. The calculated displacement maps can be saved and stored for computational correction of imaging data in further experiments. Thus, in some embodiments, previously generated displacement maps can be retrieved from a memory or other data storage device or medium and used to correct for geometrical distortions.

As another example preprocessing step, before each imaging session a background frame can be captured and saved as an average of at least 100 frames captured with the object light blocked with a shutter just before the imaging objective. The intensity values of the background frame(s) are subtracted from the acquired image data, to reduce the effects of fixed pattern noise, which may otherwise be visible in the raw images.

From the acquired image data, one or more images are reconstructed, as indicated at step 706. As an example, intensity and phase images can be reconstructed using standard procedures used in phase-stepped interferometry. It can be assumed that an image is formed through the interference of light returned from the object (“O”) and reference (“R”) on the camera, as outlined in Eqn. (2):

Iₙ=CₙO²+DₙR²+2OR|γ(δ)|\sqrt{CD} cos(ϕ+n) (2);

where Iₙ is the recorded intensity of the signal at each pixel; n indicates the phase stepped image number; C and D are the transmission coefficients for the object and reference light, respectively; γ(δ) is the envelope of the interference signal as a function of optical path mismatch; and ϕ is the optical phase step between the phase-stepped images. The coherence-gated images of the amplitude, A, and phase, Φ, components can be reconstructed using Eqns. (3) and (4), respectively:

A=\sqrt{(I₃−I₁)²+(I₄−I₂)²} (3);

$$Φ = arctan ( \frac{{I}_{4} - {I}_{2}}{{I}_{3} - {I}_{1}} ) \qquad ( 4 )$$

Additionally, in some embodiments, a reflective grid can be used to measure phase stability. For example, a sequence of frames (e.g., 1000 frames) can be captured over a period of time (e.g., 2 seconds) and the phase drift at a single pixel can be measured. In many situations, global phase drift caused by mechanical instabilities of the system can be measured and then compensated, as it affects the phase of the whole image frame in the same way.

The reconstructed images can then be displayed to a user or stored for later use, as indicated at step 708. For example, the reconstructed phase images can be further processed to create maps that depict the optical path delay induced by the sample (e.g., cells being imaged). For instance, a phase image can be unwrapped and flattened to produce quantitative images (or maps) that indicate the optical path delay induced by a sample.

While a four-phase shifting strategy to retrieve amplitude and phase information is described in the embodiments above, in some other embodiments as few as three phase-shifted images may be used for the same operation. The use of four phase shifts leads to a better SNR; however, the redundant channel may be used to harness different types of information, such as spectroscopic measurements, for dual color ratiometric imaging, or additional polarization information. Furthermore, other modalities may be implemented, such as dual-wavelength interferometry for unambiguous phase extraction, or for simultaneous two color imaging.

A benefit, which is inherent to single-shot phase retrieval, is the resilience of the phase measurements to the fluctuations in the illumination intensity. If the interferograms are captured sequentially, the temporal illumination variance has a similar effect as small random phase shifts during the pixel-wise phase fitting step. This noise is particularly detrimental for dynamic OCM measurements. In the single-shot implementations described in the present disclosure, all channels can be exposed at the same time and changes of intensity can affect all four points used for phase fitting, resulting in stable reconstructions even if the intensity fluctuates over time. This feature is particularly advantageous when using low-cost light sources, for example, with relatively noisy power supplies.

Embodiments of the present disclosure may enable new applications in biomedical research, as they can provide a new form of label-free contrast that contains information about the unperturbed dynamics of cell organelles at the subcellular level. Coherence gating permits dynamic contrast from unlabeled cells located in their native environment, in cell cultures and in tissues. In embodiments, the method may be applied for measuring and comparing tissue responses at the sub-cellular level during different pharmaceutical treatments. Also, the dynamic contrast alone can be useful for functionally phenotyping cells, such as describing the metabolic activity of different cell phenotypes for bioreactor research. Another research field that will benefit from the high speed and high sensitivity of the technology is label-free neuroimaging. Sub-nanometer level sensitivity to optical phase change can be achieved by imaging either neurons directly, or the substrates on which the cultures of neural cells are established. Cumulative phase of the light modulated by neuronal activation will contain information of both the morphology of the cells and the cell network activation patterns.

Thus, systems and methods for multiphase optical coherence tomography and/or microscopy have been disclosed. Certain embodiments permit capturing four phase-shifted imaging channels on the same camera. The 4-phase shifting approach provides a robust platform for phase-sensitive coherence gated imaging. It permits imaging at speeds only limited by the camera framerate with a phase stability that is not affected by the intensity or spectral fluctuations of the light source. With this robust design and high light-efficiency, the systems and methods described in the present disclosure can enable dynamic high-resolution FF-OCM imaging.

FIGS. 4A-4C show an example of 4-phase OCM imaging. FIG. 4A shows four simultaneously captured interferograms from a flat mirror used as a sample. FIG. 4B shows the intensity profiles from the interferograms depicted in FIG. 4A, showing that the phase in each channel is shifted by the expected step of π/2 radians used in the example embodiment described above. FIG. 4C shows an example of urea crystals grown on a glass coverslip and imaged using the systems and methods described in the present disclosure. Images show one of the raw channels used for reconstruction (left), as well as the intensity (center) and phase (right) of the OCM reconstructions for two axial locations.

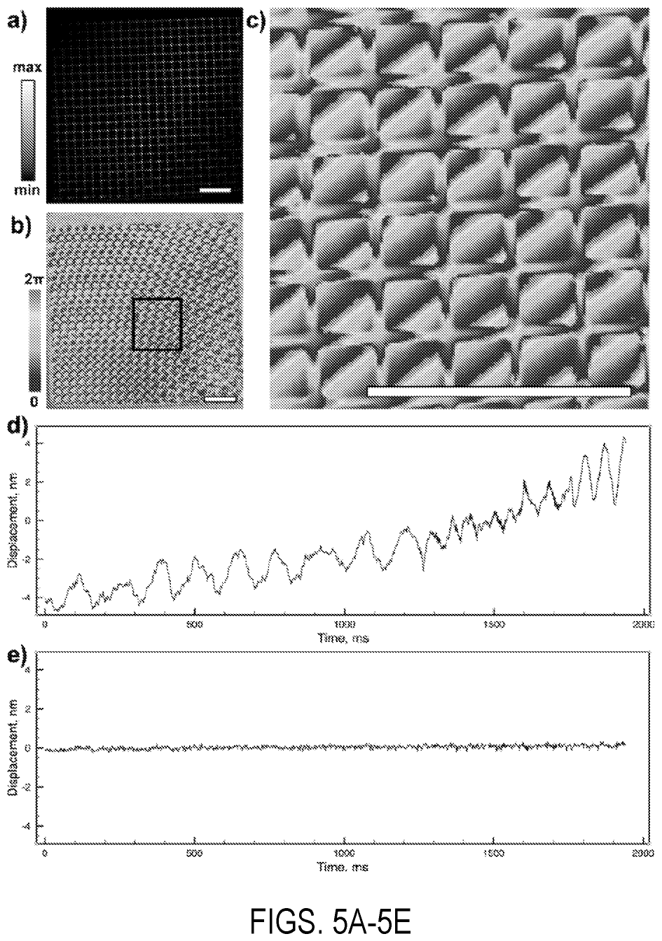

FIGS. 5A-5E show an example of phase sensitive imaging using the systems and methods described in the present disclosure. Complex images of a calibration target demonstrate high dynamic range and stability of the set-up. The images shown in FIGS. 5A, 5B, and 5C depict amplitude (FIG. 5A) and phase components (FIGS. 5B and 5C) of the image. The scale bars represent 50 μm. FIGS. 5D and 5E depict the phase stability measurements over time before and after, respectively, computational correction of global drift.

In an example study, the performance of the OCM systems described in the present disclosure was evaluated by imaging two types of cell cultures. In one sample preparation, collagen gel was prepared by using rat collagen type 1 (e.g., 3447-020-01, Millipore Sigma, St. Louis, MO, USA). A volume of 2 mL of the collagen solution in 20 mM of acetic acid with an initial concentration of 3 mg/mL was mixed with 0.3 mL of phosphate buffered saline, 25 mL of 7.5% NaHCO₃, and 0.675 mL of water for a final collagen concentration of 2 mg/mL and a pH of 7.3. All mixed solutions were kept on ice, and 0.5 mL of the solution was added to a 35 mm Petri dish with a cell adherent coating and incubated at 37° C. for 1 hour to promote gelation. Secondary cultures of NIH 3T3 mouse fibroblasts (CRL-1658, American Type Culture Collection, Manassas, VA, USA) were grown in Iscove's Modified Dulbecco's Medium with no phenol red (21056023, Thermo Fisher Scientific, Waltham, MA, USA) and supplemented with 10% v/v fetal bovine serum (16140071, Thermo Fisher Scientific, Waltham, MA, USA) and Penicillin-Streptomycin-Glutamine (10378016, Thermo Fisher Scientific, Waltham, MA, USA). Cultures were maintained in an incubator at 37° C. in an environment with 95% air and 5% CO₂, were seeded in the collagen gel, and grown overnight. The cells were imaged at room temperature within 30 minutes of being removed from the incubator.

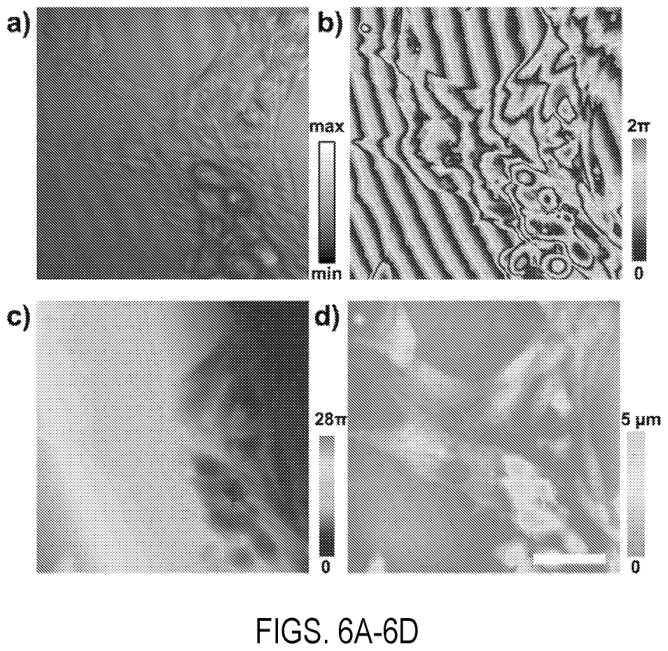

The surface of a collagen matrix, on which murine fibroblast cells were cultured, was imaged. This imaging configuration allowed for observing the cumulative phase change of light as it travelled through the cells, permitting quantitative imaging of phase changes at a 500 Hz framerate. FIGS. 6A and 6B show representative intensity and phase reconstructions. A relatively smooth phase profile of the sample permits phase unwrapping and flattening, as shown in FIGS. 6C and 6D.

In another sample preparation, B16-F10 murine melanoma cells (e.g., ATCC CRL-6475) and murine melanoma cells (ATCC CRL-6475) and murine macrophage cells J774A.1 (e.g., ATCC TIB-67) were cultured in an environment with 95% air and 5% CO₂ in Iscove's Modified Dulbecco's Medium with no phenol red (e.g., 21056023, Thermo Fisher Scientific, Waltham, MA, USA) and supplemented with 10% v/v fetal bovine serum (e.g., 16140071, Thermo Fisher Scientific, Waltham, MA, USA) and Penicillin-Streptomycin-Glutamine (e.g., 10378016, Thermo Fisher Scientific, Waltham, MA, USA). The cells were imaged at room temperature within 30 minutes of being removed from the incubator.

Inherently stable phase detection permits dynamic FF-OCM imaging of intracellular structures in murine macrophages (FIGS. 7A-7E). FIG. 7A shows an average of intensity of OCM reconstructions, FIG. 7B shows the average temporal frequency content (e.g., average of pixel-wise FFT with DC component excluded), and FIG. 7C shows the variance of the frequency content. Typically, long sequences of OCM images are advantageous for dynamic imaging because imaging contrast depends on the magnitude of the displacement of imaged structures, and longer integration times can lead to excellent contrast between the nuclei and cytoplasm of cells. However, during longer acquisitions, subcellular features can be washed out and may not be able to be resolved. Using the systems and methods described in the present disclosure, dynamic OCM imaging can be achieved. Shorter image sequences (e.g., 500 frames captured at 500 Hz framerate) permit resolving of individual parts of the cytoskeleton (FIG. 7D), while longer image sequences (e.g., 500 frames captured at 1 Hz framerate) allow for better contrast between the nucleus and cytoplasm (FIG. 7E). FIGS. 7D and 7E are cropped image subregions as indicated by the white square in FIG. 7C. The scale bar in FIGS. 7C and 7E represents 20 μm.

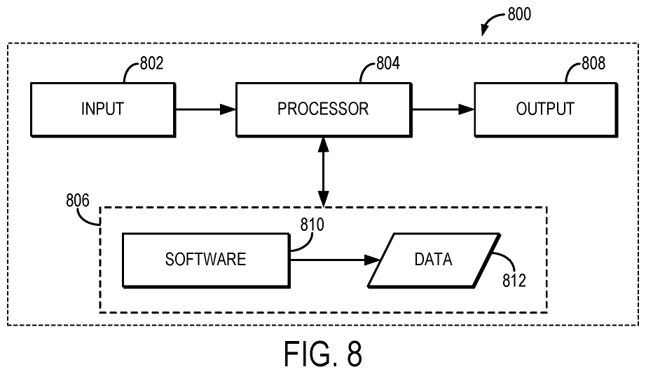

Referring now to FIG. 8, a block diagram of an example of a computer system 800 that can perform the methods of data acquisition, data preprocessing, and image reconstruction described in the present disclosure is shown. The computer system 800 generally includes an input 802, at least one hardware processor 804, a memory 806, and an output 808. Thus, the computer system 800 is generally implemented with a hardware processor 804 and a memory 806.

In some embodiments, the computer system 800 can be a workstation, a notebook computer, a tablet device, a mobile device, a multimedia device, a network server, a mainframe, one or more controllers, one or more microcontrollers, or any other general-purpose or application-specific computing device.

The computer system 800 may operate autonomously or semi-autonomously, or may read executable software instructions from the memory 806 or a computer-readable medium (e.g., a hard drive, a CD-ROM, flash memory), or may receive instructions via the input 802 from a user, or any another source logically connected to a computer or device, such as another networked computer or server. Thus, in some embodiments, the computer system 800 can also include any suitable device for reading computer-readable storage media.

In general, the computer system 800 is programmed or otherwise configured to implement the methods and algorithms described in the present disclosure. For instance, the computer system 800 can be programmed to acquire image data using an image sensor, preprocess the image data, and reconstruct one or more images from the acquired image data, as described above in more detail.

The input 802 may take any suitable shape or form, as desired, for operation of the computer system 800, including the ability for selecting, entering, or otherwise specifying parameters consistent with performing tasks, processing data, or operating the computer system 800. In some aspects, the input 802 may be configured to receive data, such as data acquired with an image sensor of a multiphase optical coherence tomography or multiphase optical coherence microscopy system. Such data may be processed as described above to reconstruct one or more images (e.g., amplitude and/or phase images).

Among the processing tasks for operating the computer system 800, the one or more hardware processors 804 may also be configured to carry out any number of post-processing steps on data received by way of the input 802.

The memory 806 may contain software 810 and data 812, such as data acquired with a multiphase optical coherence tomography and/or multiphase optical coherence microscopy system, and may be configured for storage and retrieval of processed information, instructions, and data to be processed by the one or more hardware processors 804. In some aspects, the software 810 may contain instructions directed to reconstructing images as described above.

In addition, the output 808 may take any shape or form, as desired, and may be configured for displaying images reconstructed using the systems and methods described in the present disclosure, in addition to other desired information.

The present disclosure has described one or more preferred embodiments, and it should be appreciated that many equivalents, alternatives, variations, and modifications, aside from those expressly stated, are possible and within the scope of the invention.

1. An optical coherence microscopy system, comprising:
a light source configured to produce a light beam, wherein the light beam comprises S-polarized light and P-polarized light;
a beamsplitter configured to split the light beam into a reference beam and an object beam, wherein the reference beam comprises an S-polarized reference beam component (RS) and a P-polarized reference beam component (RP) and the object beam comprises an S-polarized object beam component (OS) and a P-polarized object beam component (OP);
a reference arm configured to receive the reference beam from the beamsplitter, impart a phase shift to at least one of the S-polarized reference beam component or the P-polarized reference beam component, and return the reference beam to the beamsplitter;
an object arm configured to receive the object beam from the beamsplitter and return the object beam to the beamsplitter; and
wherein the beamsplitter is further configured to combine the reference beam returned from the reference arm with the object beam returned from the object arm to output a phase-shifted light beam comprising a plurality of phase-shifted light beam components.

2. The optical coherence microscopy system of claim 1, wherein the phase-shifted light beam components comprise multiple distinct phase shifts between the reference beam returned from the reference arm and the object beam returned from the object arm.

3. The optical coherence microscopy system of claim 2, wherein the multiple distinct phase shifts are stepped by π/2.

4. The optical coherence microscopy system of claim 2, wherein the phase-shifted light beam components comprise four distinct phase shifts.

5. The optical coherence microscopy system of claim 1, wherein the beamsplitter is a non-polarizing beamsplitter.

6. The optical coherence microscopy system of claim 1, wherein the reference arm comprises:
a phase shifter that shifts a phase of at least one of the S-polarized reference beam component or the P-polarized reference beam component; and
a reference reflector that reflects the reference beam received from the beamsplitter back onto the beamsplitter;
wherein the reference beam firstly passes through the phase shifter, is reflected by the reference reflector, and secondly passes through the phase shifter before exiting the reference arm.

7. The optical coherence microscopy system of claim 6, wherein the phase shifter comprises a waveplate.

8. The optical coherence microscopy system of claim 7, wherein the waveplate is a λ/8 waveplate.

9. The optical coherence microscopy system of claim 7, wherein the waveplate is an achromatic waveplate.

10. The optical coherence microscopy system of claim 6, wherein the reference reflector comprises a reference mirror.

11. The optical coherence microscopy system of claim 1, wherein the beamsplitter is configured to impart an additional phase shift to light reflected by the beamsplitter.

12. The optical coherence microscopy system of claim 11, wherein the beamsplitter is configured to impart an additional π phase shift to light reflected by the beamsplitter.

13. The optical coherence microscopy system of claim 11, wherein the beamsplitter is configured to:
firstly reflect the object beam when splitting the object beam into the object arm, thereby imparting the additional phase shift to the object beam; and
firstly transmit the reference beam when splitting the reference beam into the reference arm, thereby not imparting the additional phase shift to the reference beam.

14. The optical coherence microscopy system of claim 13, wherein the beamsplitter is configured to:
split the object beam returned from the object arm into a first object beam part and a second object beam part, wherein the first object beam part is transmitted by the beamsplitter and the second object beam part is reflected by the beamsplitter thereby accruing the additional phase shift; and
split the reference beam returned from the reference arm into a first reference beam part and a second reference beam part, wherein the first reference beam part is reflected by the beam splitter thereby accruing the additional phase shift and the second reference beam part is transmitted by the beamsplitter.

15. The optical coherence microscopy system of claim 1, wherein the light source is an unpolarized light source.

16. The optical coherence microscopy system of claim 1, wherein the light source comprises a light emitting diode (LED).

17. An optical assembly for use in an interferometer, comprising:
a beamsplitter configured to extract a reference beam from a light beam when the light beam firstly propagates through the beamsplitter;
a reference arm configured to receive the reference beam and impart a phase shift thereto before returning the reference beam to the beamsplitter, wherein the reference arm comprises a phase shifter and a reference reflector; and
wherein the beamsplitter integrates the reference beam returned from the reference arm into the light beam when the reference beam propagates through the beamsplitter.

18. The optical assembly of claim 17, wherein the phase shifter is a waveplate and the reference arm is configured such that the reference beam firstly passes through the waveplate, is reflected by the reference reflector, and secondly passes through the waveplate before exiting the reference arm.

19. The optical assembly of claim 18, wherein the phase shift comprises a first phase shift imparted when the reference beam firstly passes through the waveplate and a second phase shift imparted when the reference beam secondly passes through the waveplate.

20. The optical assembly of claim 17, wherein the beamsplitter is further configured to extract an object beam from the light beam when the light beam firstly propagates through the beamsplitter, and wherein the optical assembly further comprises an object arm configured to receive the object beam and to return a reflected object beam;
wherein the beamsplitter integrates the object beam into the light beam when the reflected object beam propagates through the beamsplitter.

21. The optical assembly of claim 17, wherein the beamsplitter is configured to impart an additional phase shift to the reference beam when the reference beam is reflected by the beamsplitter.

22. The optical assembly of claim 17, wherein the beamsplitter is a non-polarizing beamsplitter.

23. A method for multiphase optical coherence microscopy, comprising:
extracting a reference beam and an object beam from a light beam, wherein the object beam comprises a P-polarized object beam component (OP) and an S-polarized object beam component (OS), and the reference beam comprises a P-polarized reference beam component (RP) and an S-polarized reference beam component (RS);
shifting a phase of at least one of the RS component or the RP component of the reference beam; and
recombining the reference beam and the object beam to obtain distinct phase shifts between the object beam and the reference beam in at least one of the S-polarization or P-polarization components.

24. The method of claim 23, wherein the distinct phase shifts comprise distinct phase shifts stepped by π/2 between the object and the reference beams in at least one of the S-polarization or P-polarization components.
